(19) United States
(12) Patent Application Publication
(10) Pub. No.: US 2013/0118488 A1
(43) Pub. Date: May 16, 2013

(54) Nasal Therapeutic System

(71) Applicant: Barbara Ledogar, East Setauket, NY (US)

(72) Inventor: Barbara Ledogar, East Setauket, NY (US)

(21) Appl. No.: 13/295,212

(22) Filed: Nov. 14, 2011

(51) Int. Cl. A61F 5/08; A61M 16/00

(52) U.S. Cl. 128/203.12

(57) ABSTRACT

A nasal therapeutic system comprising a nasal dilator that prevents the outer wall tissue of the nasal passages of the nose from drawing in during breathing having a truss member with an aromatherapy means, which includes a flexible strip of material having a plurality of regions, and an intermediate segment, the regions are adapted to engage the outer wall tissue of nasal passages of the nose, wherein the intermediate segment is configured to traverse a portion of a nose located between the first and second nasal passages, and a pad can be provided to prevent and/or limit adhesion of the intermediate segment to the nose, wherein the truss member further includes resilient bands secured to the strip of material adjacent opposite edges of the intermediate segment, to stabilize the outer wall tissue and thereby prevent the outer wall tissue of the nasal passages from drawing in.

### BACKGROUND OF THE INVENTION

This invention relates to a nasal therapeutic system, in particular, an improved nasal therapeutic system device that provides, inter alia, a system which maintains and/or maximizes the cross-sectional pathway of the nose at rest or during consciousness, while providing aromatherapy.

Historically, some consumers suffer from a malformation of the nasal passages which makes breathing difficult, e.g., a deviated septum and/or swelling due to allergies. The lower portion of the nostril, immediately above the entrance to the nostril, is known as a vestibule, which tapers inwardly to a narrowed neck-like area called the ostium internum, wherein above the ostium internum the nasal passages widen out again. Nasal obstructions commonly occur at the ostium in individuals who have swelling due to allergic reactions, a deviated septum, or similar condition, to the point that the ostium may be substantially blocked. Commonly, the lateral wall (i.e., the outer wall tissue of the nasal passage) at the ostium is loose with the result that the outer wall tissue draws in during the process of inhalation to substantially block the passage of air through the nasal passage. The drawing in of the outer wall tissue act as a “check valve” to block air flow during in-breathing.

Blockage of the nasal passages is obviously an inconvenience to anyone who experiences it. In particular, sustained mouth breathing over a long period of time may cause lung irritation due to the inhalation of foreign particles that would otherwise be filtered if the breath had been passed through the nose, which is not uncommon for individuals with asthma, which further may lead to lung infections and enlarged vocal cords. Blockage of the nasal passages is particularly uncomfortable at night, since it is difficult for a person who has such a problem to breathe through the mouth while asleep. Nasal blockage can lead to sleep disturbances and irregularities, since a person with such a condition may wake often because they are not inhaling sufficient quantities of oxygen, thereby may lead to low oxygen count, and hence wake up feeling tired, and may eventually never seem to get enough rest, causing long term fatigue.

One approach to remedy the serious and chronic nasal blockage problem as described above is a surgical attempt to correct the malformation of the nasal passages. However, surgery is expensive in terms of time and money, and may not ultimately correct the problem to the satisfaction of the patient.

As a surgical alternative, nasal dilators have been used for aiding breathing through the nose e.g., U.S. Pat. No. 4,414,977 to Rezakhany discloses a nasal dilator, having a generally elongated top and bottom rings which are spaced apart and connected together by a rear strut and a front strut. The front strut is longer than the rear strut and includes a bend therein formed at a position close to the front end of the bottom ring. When in place in the nasal passage, the top ring fits in the ostium within the nostril to prevent the tissue from being dram in during inhalation, and to reduce extra flow resistance during exhalation. The bottom ring fits above the entrance to the nostril and serves to stabilize the position of the top ring within the nasal passage. One of these nasal dilators must be inserted into each nasal passage to provide unobstructed breathing.

However, these nasal dilators are not always effective and they are uncomfortable to wear, because the nasal dilators must be inserted within the nasal passages they may cause irritation and itching. In addition, these nasal dilators must be custom-made to fit each nasal passage of an individual, which may be cost prohibitive.

Other external nasal dilators include those in U.S. Pat. No. 1,292,083 to Sawyer and U.S. Pat. No. 1,950,839 to Chirila which attach to the outer wall tissues of the nasal passages to pull outwardly thereon. Such external dilators have been inconvenient and cumbersome due in part of multiple parts requiring adjustment to operate together satisfactorily and bearing the risk of unwanted disassembly due to disturbance forces encountered during use.

Hence there is a long standing need for improved nasal dilators for preventing outer wall tissue of nasal passages of a nose from drawing in during breathing, without the invasiveness of inserting an object within the nasal passage.

Moreover, there is a need for a nasal dilator that can be worn at night when the nasal blockage problem is most acute and most uncomfortable and external disturbance forces are most often encountered as a result of movement head and pillow movement during hours intended for sleeping, wherein any such dilator should be of efficient design and relatively uncomplicated and provide effective stabilization of the outer wall tissue of the nasal passages to provide effective relief from nasal blockage during inhalation, whilst providing this effective stabilization without undue discomfort or risk to the wearer.

Moreover, aromatherapy is the use of essential oils from plants for healing, for example, eucalyptus or menthol. Whether inhaled or applied on the skin, essential oils are gaining new attention as an alternative treatment for infections, stress, and other health problems. Essential oils are concentrated extracts taken from the roots, leaves, seeds, or blossoms of plants. Each contains its own mix of active ingredients, and this mix determines what the oil is used for. Some oils are used to promote physical healing e.g., to treat swelling or fungal infections, whilst others are used for their emotional value e.g., they may enhance relaxation or make a room smell pleasant. Orange blossom oil, for example, contains a large amount of an active ingredient that is thought to be calming.

Essential oils have been used for therapeutic purposes for nearly 6,000 years, wherein the ancient Chinese, Indians, Egyptians, Greeks, and Romans used them in cosmetics, perfumes, and drugs. These essential oils were also commonly used for spiritual, therapeutic, hygienic, and ritualistic purposes.

However, no one has combined the benefits of both the nasal device with aromatherapy, and hence the need for the same. More specifically, by combining the two, portability is increased, and the consumer can experience the benefit of both any time, and any place.

### SUMMARY OF THE INVENTION

The present invention is a nasal therapeutic system for preventing outer wall tissue of nasal passages of a nose from drawing in during breathing. The nasal therapeutic system comprises a truss member having a first end region adapted to engage the outer wall tissue of a first nasal passage. A second end region of the truss member is configured to engage the outer wall tissue of a second nasal passage. The first and second end regions of the truss member are coupled to one another by an intermediate segment. The intermediate segment is configured to traverse a portion of the nose located between the first and second nasal passages. The truss member, when in place, acts to stabilize the outer wall tissue and thereby prevent the outer wall tissue of the first and second nasal passages from drawing in during breathing.

The truss member includes a flexible strip of material that defines the first and second end regions and the intermediate segment of nasal therapeutic system, with an aromatherapy means. A first resilient band is secured to a first side of the strip of material adjacent a first edge of the material. A second resilient band spaced from the first resilient band is secured to the first side of the strip of material adjacent a second edge thereof. The first and second resilient bands are oriented generally parallel to one another and substantially parallel to the longitudinal extent of the strip of material.

Each of the first and second resilient bands includes a plurality of grooves that extend substantially parallel to the respective resilient band. The grooves create areas of reduced material to enhance the flexibility of the first and second resilient bands in a direction perpendicular to the grooves. In addition, each of the first and second resilient bands includes first and second angled ends. The first and second angled ends extend towards the first side of the strip of material and help to prevent the first and second resilient bands from readily separating from the strip of material when the truss member is flexed. The resiliency of the first and second resilient bands prevent the outer wall tissue of the first and second nasal passages from drawing in during breathing.

The truss member further includes an adhesive substance located on a second side of the flexible strip of material. The adhesive substance acts to releasably secure the truss member to the outer wall tissue of the first and second nasal passages, particularly the adhesive substance portions on the surfaces near the ends of the second side of the flexible strip of material in the truss member. An optional pad at a surface intermediate to these end surfaces can be used to prevent adhesion to the bridge of the wearer's nose for added convenience and comfort. Alternatively, rather than such a pad, another optional arrangement can be used at this intermediate surface permitting partial adhesion to the bridge of the wearer's nose for greater comfort. First and second release liners cover the adhesive substance on the first and second end regions. The first and second release liners are readily removable from the strip of material to expose the adhesive substance and permit the truss member to be secured to the outer wall tissue of the first and second nasal passages.

This nasal therapeutic system is of efficient design and effectively prevents the outer wall tissue of the first and second nasal passages of the nose from drawing in during breathing. In addition, the nasal therapeutic system provides effective relief of nasal blockage during inhalation without the irritation and discomfort normally associated with nasal therapeutic systems that are inserted within the nasal passages. Moreover, this nasal therapeutic system can be worn at night when the inhalation nasal blockage problem is most acute, without the anxiety and inconvenience normally associated with custom made, internally worn nasal dilators or multiple and separable part external dilators.

Other objectives, advantages, and novel features of the present invention will become more apparent from the following detailed description when taken in conjunction with the accompanying drawings.

### BRIEF DESCRIPTION OF THE DRAWINGS

FIG. 1 is perspective view of a portion of a face with a nasal therapeutic system in accordance with the present invention secured to a nose.

FIG. 2 is an exploded perspective view showing the components of the nasal therapeutic system in accordance with the present invention.

FIG. 3 is a perspective view similar to FIG. 1 with the nasal therapeutic system in accordance with the present invention removed from the nose.

FIG. 4 is a sectional view taken along line 4-4 in FIG. 3 showing the nose in a state wherein no appreciable flow of air is occurring in the nasal passages.

FIG. 5 is a sectional view similar to FIG. 4 showing the state of the nose during inhalation.

FIG. 6 is a sectional view taken along line 6-6 in FIG. 1 showing the state of the nose during inhalation with the nasal therapeutic system in accordance with the present invention secured thereto.

FIG. 7 is an exploded perspective view showing the components of an alternative nasal therapeutic system in accordance with the present invention.

FIG. 8 is a fragmentary view of an alternative for a portion of the nasal therapeutic system shown in FIG. 7.

FIG. 9 is a fragmentary view of another alternative for a portion of the nasal therapeutic system shown in FIG. 7.

FIG. 10 is a fragmentary view of a further alternative for a portion of the nasal therapeutic system shown in FIG. 7.

FIG. 11 is a fragmentary view of yet another alternative for a portion of the nasal therapeutic system shown in FIG. 7.

### DETAILED DESCRIPTION OF THE INVENTION

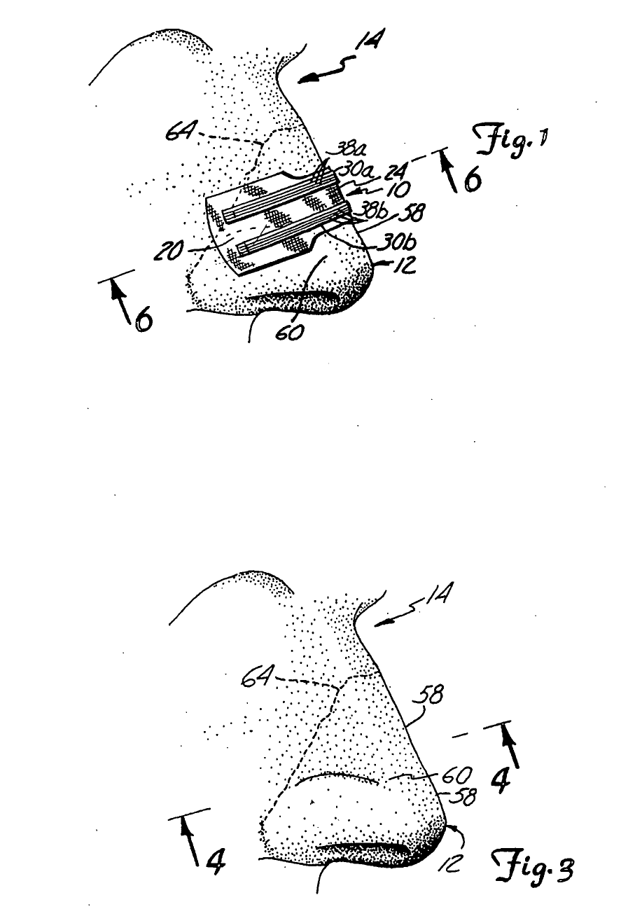

The following descriptions of the preferred embodiments are presented to illustrate the present invention and are not to be construed to limit the claims in any manner whatsoever. In reference to the drawings, namely FIGS. 1 to 11, the preferred embodiment of the present invention is disclosed, which is directed to A nasal therapeutic system 10 in accordance with the present invention is illustrated generally in FIG. 1. The nasal therapeutic system 10 is shown secured to a nose 12 of a wearer 14.

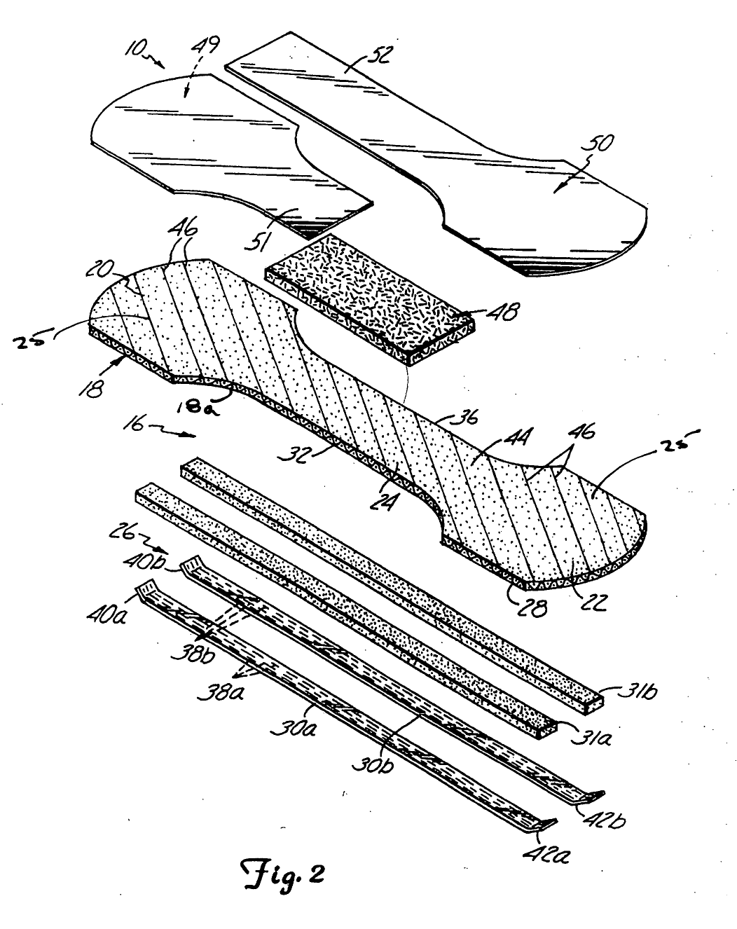

As seen in FIG. 2, the nasal therapeutic system 10 comprises a truss member 16 including a flexible strip of material 18 having a first end region 20 and a second end region 22 coupled to the first end region 20 by way of an intermediate segment 24. The width of the intermediate segment 24 is less than the width of the first and second end regions 20 and 22. The flexible strip of material 18 is preferably formed of an interwoven piece of fabric that allows the skin of the nose 12 to breathe to maximize comfort and minimize irritation. As an alternative, the strip of material 18 may be formed of a plastic film.

The truss 16 member further includes resilient means 26 secured to a first side 28 of the strip of material 18. The resilient means 26 includes a first resilient band 30a secured by a first adhesive member 31a to the first side 28 of the strip of material 18. The first resilient band 30a is secured to the strip of material 18 adjacent a first edge 32 of the intermediate segment 24. In addition, a second resilient band 30b, spaced from the first resilient band 30a, is secured by a second adhesive member 31b to the first side 28 of the strip of material 18. The second resilient band 30b is secured to the strip of material 18 adjacent a second edge 36 of the intermediate segment 24. The first and, second resilient bands 30a and 30b are oriented generally parallel to one another and substantially Parallel to the longitudinal extent of the flexible strip of material 18. Each of the first and second adhesive members 31a and 31b is formed of an adhesive material such as double sided adhesive, foam tape. Said system 10 further including an aromatherapy means 18a, wherein flexible strip 18 is infused and/or impregnated with an essential oil; and/or wherein said means 18a may be limited to only the first side 28 of the flexible strip 18 if the strength of the desired essential oil is too strong.

Each of the first and second resilient bands 30a and 30b includes a plurality of grooves 38a and 38b, respectively, that extend substantially parallel to the respective resilient band 30a and 30b. As seen best in FIG. 2, the grooves 38a and 38b are formed in the exposed sides of the first and second resilient bands 30a and 30b (i.e., the sides of the first and second resilient bands 30a and 30b opposite that to which the first and second adhesive members 31a and 31b are secured). The grooves 38a and 38b create areas of reduced material to enhance the flexibility of the first and second resilient bands 30a and 30b in a direction perpendicular to the plurality of grooves 38a and 38b. In addition, each of the first and second resilient bands 30a and 30b includes first angled ends 40a and 40b, respectively, and second angled ends 42a and 42b, respectively. The first and second angled ends 40a,b and 42a,b extend towards the first side 28 of the strip of material 18 and help to prevent the first and second resilient bands 30a and 30b from readily separating from the strip of material 18 and the first and second adhesive members 31a and 31b when the truss member 10 is flexed. The first and second resilient bands 30a and 30b are formed of a plastic material.

As seen in FIG. 2, a second side 44 of the strip of material 18 includes a layer of an adhesive substance 46 that extends over the first and second end regions 20 and 22 and the intermediate segment 24. The adhesive substance 46 is biocompatible with the skin of the nose 12, wherein said segment 24 may further include a moisturizer, such as aloe 25, so as to minimize damage to the skin before, during, and post use. For example, aloe may be included on said side 44. A padded element 48 is secured to the median of the intermediate segment 24 via the adhesive substance 46. Readily removable, first and second release liners 49 and 50, respectively, cover the adhesive substance 46 on the first and second end regions 20 and 22, respectively, of the strip of material 18. The first and second release liners 49 and 50 cover the adhesive substance 46 and remain in place on the strip of material 18 until the nasal therapeutic system 10 is to be deployed. The first and second release liners 49 and 50 also include extensions 51 and 52, respectively, that cover the padded element 48 and further act to protect the padded element 48 until the nasal therapeutic system 10 is to be secured to the nose 12 of a wearer 14.

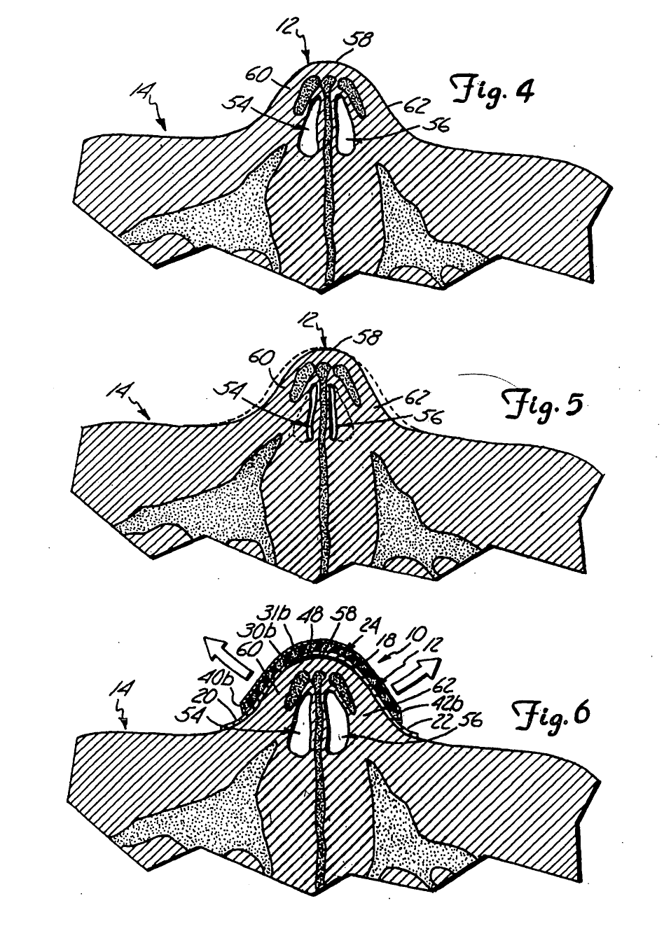

As seen in FIGS. 3 and 4, the nose 12 includes a first nasal passage 54, a second nasal passages 56 and a portion of the nose 12 known as the bridge 58 located between the first and second nasal passages 54 and 56. FIG. 4 illustrates the state of the first and second nasal passages 54 and 56 when no appreciable flow of air is occurring through the nasal passages 54 and 56. Due to a malformation, such as a deviated septum or swelling due to allergic reactions, outer wall tissue 60 and 62 of the first and second nasal passages 54 and 56, respectively, tends to be drawn in (i.e., collapse) during inhalation (see FIG. 5). This drawing in during inhalation is caused by reduced air pressure within the first and second nasal passages 54 and 56 as a result of an increase in air velocity as the in drawn breath travels through the first and second nasal passages 54 and 56. The portion (i.e., the ostium) of the outer wall tissue 60 and 62 drawn in during inhalation is that located between the nasal cartilage 64 (shown in dashed lines in FIGS. 1 and 3) and the entrance to the nasal passages 54 and 56. This drawing in of the outer wall tissue 60 and 62 causes nasal blockage. The nasal therapeutic system 10 of the present invention remedies this problem.

To secure the nasal therapeutic system 10 to the nose 12, the first and second release liners 49 and 50 are removed from the flexible strip of material 18 to expose the adhesive substance 46. As seen in FIGS. 1 and 6, the nasal therapeutic system 10 is placed on the exterior of the nose 12 such that the intermediate segment 24 traverses the bridge 58 of the nose 12 and the first and second end regions 20 and 22 contact the outer wall tissue 60 and 62 of the first and second nasal passages 54 and 56. The adhesive substance 46 on the first and second end regions 20 and 22 releasably secures the truss member 16 to the outer wall tissue 60 and 62 of the first and second nasal passages 54 and 56. As seen in FIG. 6, the padded element 48 creates an absorbent adhesive void between the truss member 16 and the bridge 58. This absorbent adhesive void absorbs moisture due to perspiration or the like. With the nasal therapeutic system 10 in place about the nose 12, the resiliency of the first and second resilient bands 30a and 30b (i.e., the tendency of the resilient bands to return to their normally planar state shown in FIG. 2) acts to stabilize the outer wall tissue 60 and 62 and thereby prevents the outer wall tissue 60 and 62 of the first and second nasal passages 54 and 56 from drawing in during breathing (i.e., during inhalation). In addition, the flexibility of the strip of material 18 and the first and second adhesive members 31a and 31b, the resiliency of the first and second bands 30a and 30b, and the flexibility of the first and second bands 30a and 30b due to the grooves 38a and 38b, all allow the nasal therapeutic system 10 to closely conform to the curves of the nose of each individual wearer.

This nasal therapeutic system 10 is of efficient design and effectively prevents the outer wall tissue 60 and 62 of the first and second nasal passages 54 and 56 of the nose 12 from drawing in during breathing. In addition, the nasal therapeutic system 10 provides effective relief of nasal blockage during inhalation without the irritation and discomfort normally associated with nasal dilators that are inserted within the nasal passages. Moreover, this nasal therapeutic system 10 can be worn at night when the inhalation nasal blockage problem is most acute, without the anxiety and inconvenience normally associated with custom made, internally worn nasal dilators.

The adhesive void at bridge 58 of nose 12 of user 14 formed by the use of padded element 48 to cover a portion of adhesive 46 can, for some users, cause irritation. Such irritation is thought to arise from relative movements between padded element 48 and the skin at bridge 58 of nose 12, movements which come about because of facial muscles causing movement of that skin or because of external objects, such as a pillow, applying forces to nasal therapeutic system 10 at various times during use. This problem can be avoided, of course, by not using pad element 48 so that adhesive 46 adheres to all of bridge 58 and outer wall tissues 60 and 62 of nose 12. Such an omission of pad element 48, on the other hand, eliminates the benefits derived from its use such as the avoidance of peel forces on the skin of bridge 58 during removal of nasal therapeutic system 10 after use.

An alternative which results in providing some of the benefits of both a fully adhesive nasal therapeutic system and a nasal therapeutic system with an intermediate adhesive void can be provided by forming a nasal therapeutic system having a partial adhesive void at the intermediate location between the ends thereof. Such a partial adhesive void can be provided through having the adhesive present being effective in adhering to the skin of the bridge of the user's nose only over some fraction of the intermediate surface area between the end surfaces of the nasal therapeutic system. In that intermediate segment that goes over bridge 58 of nose 12 of user 14, a typical choice of effective adhesion area would be 40 to 50% of the entire nasal therapeutic system intermediate surface area for a biocompatible adhesive material typically used for adhesive substance 46. The extent of the reduction in adhesion at the skin of bridge 58 and nose 12 by a partial adhesive void can be conveniently and consistently determined by the choice of the fraction of the intermediate area in which the adhesive is effective, a choice which can be made and maintained in the manufacturing process for nasal therapeutic system 10.

The fractional reduction of the effective adhesive area from the total intermediate surface area covering bridge 58 of nose 12 during use permits sufficient adhesion to avoid most of the irritation occurring due to relative motion of the skin on bridge 58 and nasal therapeutic system 10, but yet provides reduced peel forces during removal of nasal therapeutic system 10 from nose 12 of user 14 after completion of the use thereof. In such an arrangement, there will be no reduction in the adhesion force which occurs between the ends of nasal therapeutic system 10 and wall tissues 60 and 62 of nose 12. As a result, the outward spring force of nasal therapeutic system 10 will be balanced by the reactive force of wall tissues 60 and 62 without separation of the ends of nasal therapeutic system 10 from those tissues to thereby maintain wider nasal passages adjacent such wall tissues.

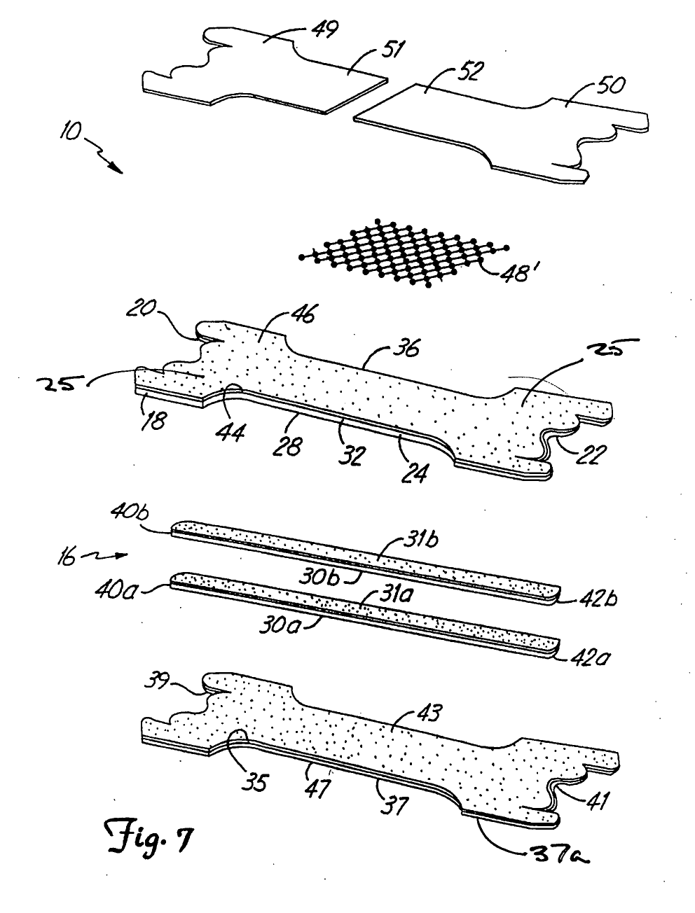

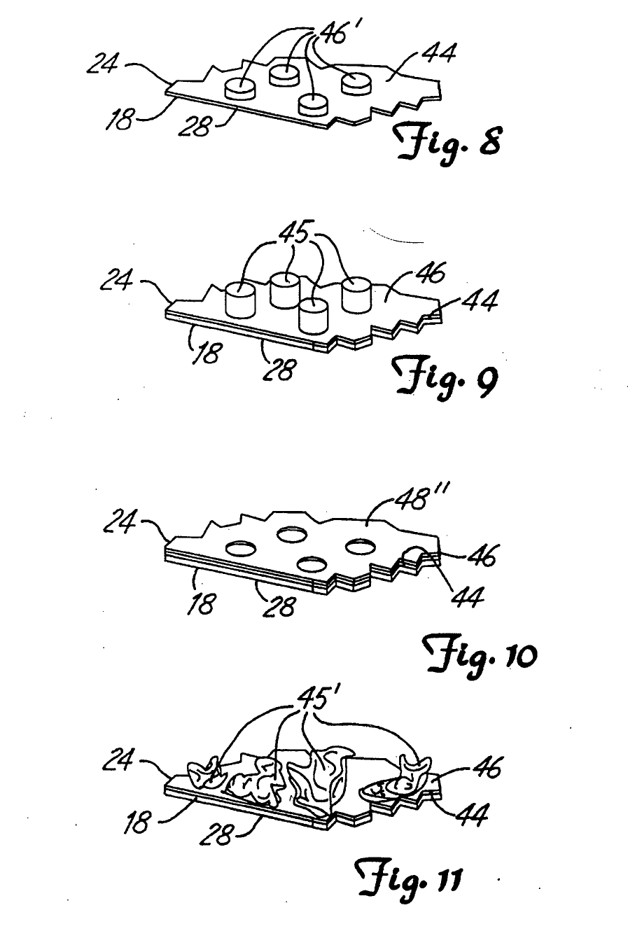

FIG. 7 illustrates an exploded perspective view of nasal therapeutic system 10 having a structure providing a partial adhesive void in an intermediate location between the end surfaces thereof, wherein system 10 comprises unitary, or single body, truss member 16 which includes flexible strip of material 18 having first and second end regions 20 and 22 coupled together by intermediate segment 24 as before; wherein, as before, said segment 24 may further include a moisturizer, such as aloe, so as to minimize damage to the skin before, during, and post use. For example, aloe 25 may be included on said side 44, and is most important to cover the portion of the system that ultimately contacts the skin of the wearer. First and second end regions 20 and 22 are each shown with a scalloped end shape provided between a pair of side extensions extending further outward along the length of nasal therapeutic system 10, an arrangement provided to reduce unintended peeling of nasal therapeutic system 10 from the user's nose 12 during use.

Again in truss member 16 are first and second resilient bands 30A and 30B, as before, and formed of industrial grade, biaxial oriented polyester about 0.010 inches thick and typically from 0.080 to 0.135 in. wide. These resilient bands are again secured to side 28 of flexible material strip 18 by strips of acrylic, pressure sensitive, biocompatible adhesive material 31A and 31B, but these strips are shown in FIG. 7 as homogeneous adhesive material layers coated onto these resilient bands rather than being shown separately as in FIG. 2. The ends of resilient bands 30A, 30B are designated 40a, 40b, 42a and 42b as before but are not angled in this version. The grooves used in the resilient bands in the nasal therapeutic system of FIG. 2 are omitted in the more torsionally flexible bands of the nasal therapeutic system of FIG. 7.

The side of flexible material strip 18 opposite its side 28 to which resilient bands 30A and 30B are secured, side 44, has again adhesive substance layer 46 extended thereover to cover end regions 20 and 22 and intermediate segment 24 with this substance again being a porous, acrylic, pressure sensitive, biocompatible adhesive. However, rather than providing padded element 48 on adhesive substance 46 at intermediate segment 24 as in FIG. 2, a netting or mesh layer, 48′, is instead secured to adhesive substance 46 at the location of intermediate segment 24, wherein said segment 24 may further include a moisturizer, such as aloe, so as to minimize damage to the skin before, during, and post use. For example, aloe may be included on said side 44.

Netting 48′ is a polyethylene material netting having a thickness of around 0.004 in. This netting has significant mechanical strength even when wet, and allows moisture to easily wick therealong so that it may be transmitted away from the skin on bridge 58 of user's nose 12 through the vapor porous materials thereover. The netting material should not introduce any contaminants and should be of an odorless nature. In its mechanical characteristics, the material should be as soft as, or softer than, flexible material strip 18 (a material which can be a non-woven, spun lace, 100% polyester fabric such as is available from E. I. DuPont Nemours & Co. under the trademark SONTARA®), that is, the material should drape over uneven surfaces under the influence of gravity in a manner so as to follow those surfaces as well as does flexible material strip 18. This characteristic indicates that the netting material can also compress in on itself sufficiently to prevent significant puckering of that material when forced about a curved surface over bridge 58 of user's nose 12 even though that material is fabricated in a substantially planar state. A suitable material for netting 48′ is offered under the trademark DELNET® by Applied Extrusion Technologies, Inc. of Middletown, Del.

Netting 48′ thus serves as both a partial mask in covering portions of adhesive 46 at the location of intermediate segment 24, and as a spacer between the skin of bridge 58 of user's nose 12, when in use, and portions of both adhesive material 46 and flexible material strip 18. By both covering portions of adhesive material 46 in its masking function, and by reducing the compression of adhesive 46 against the skin of bridge 58 of user's nose 12, a reduced adhesion is obtained between the skin of bridge 58 and the portion of nasal therapeutic system 10 at its intermediate segment 24. The adhesion obtained is sufficient to prevent relative movement of nasal therapeutic system 10 with respect to the skin of bridge 58 of user's nose 12 in most situations, but is sufficiently reduced to allow removal of nasal therapeutic system 10 after completion of use without experiencing the peel forces between nasal therapeutic system 10 and that skin which would otherwise occur in the absence of netting material 48′.

Unitary truss member 16 further includes a flexible strip on top, or outer, porous non-woven material, 37, with first end region 39 and second end region 41 along with intermediate segment 47. These end regions and intermediate segment of top material 37 have the same size and shape as end regions 20 and 22 of flexible material strip 18 and intermediate segment 24 thereof. A water vapor porous, acrylic, pressure sensitive, biocompatible adhesive material layer, 43, is provided on surface 35 of top material 37 to secure top material 37 to resilient bands 30A and 30B and to the exposed portions of flexible material strip 18 not covered by these resilient bands in their being secured thereto. Top material strip 37 aids in preventing the resilient bands from separating from base material 18 when nasal therapeutic system 10 is flexed around nose 12 in use, and aids in maintaining the integrity of base material 18 during removal of nasal therapeutic system 10 from the user's nose after use. Said system 10 further including an aromatherapy means 37a, wherein top material 37 is infused or impregnated with an essential oil.

Again, first and second release liners 49 and 50 cover the exposed portions of adhesive substance layer 46 over first and second end regions 20 and 22 of flexible strip of material 18. Release liners 49 and 50 are provided to cover adhesive 46 until nasal therapeutic system 10 is ready to be used at which time they are peeled off by the user to expose adhesive substance 46. First and second release liners 49 and 50 again include extensions 51 and 52 to cover netting 48′ to shield that element and the portions of adhesive 46 exposed there from the external world until nasal therapeutic system 10 is to be used.

Other arrangements can be used to provide the partial adhesive void structure desired at intermediate segment 24 rather than the use of netting material 48′ at that location as shown in FIG. 7, however, in all embodiments, an aromatherapy means is included therein. One such alternative is shown in the fragmentary view of FIG. 8 of a portion of an alternate construction for intermediate segment 24 of nasal therapeutic system 10. There, adhesive substance 46, rather than being masked by netting material 48′ is shown partially removed by etching or dissolving through mask portions thereof to leave a plurality of separated adhesive regions, 46′. Alternatively, rather than outright removal, the portions of adhesive 46 not to be effective in adhering nasal therapeutic system 10 to the skin of bridge 58 could be chemically deactivated to no longer adhere in the deactivated regions. The amount of adhesion reduction from the adhesion provided by the use of adhesive layer 46 without modification is controlled by the size of effective adhesive regions 46′ and by the separation provided between these regions. In a further alternative, separated adhesive regions 46′ could instead be additively provided rather than substractively provided such as in the form of separated parallel rows of adhesive material (or separated adhesive spots as in FIG. 8) specifically deposited on flexible material strip 18 in this form to provide the desired partial adhesive void.

FIG. 9 illustrates an alternative treatment for covering portions of adhesive substance 46, as compared to full coverage methods. Such full coverage treatments or depositions of adhesive material 46 and intermediate segment 24 of nasal therapeutic system 10 may prove relatively expensive when compared with methods involving partially covering a full layer of adhesive substance 46 at intermediate segment 24 such as occurs with use of netting 48′. FIG. 9 illustrates an alternative method for covering portions of adhesive substance 46 in the fragmentary view provided thereof a portion of intermediate segment 24 using the construction for this alternative, wherein a plurality of spaced-apart polyethylene disks or beads, 45, are shown adhered to adhesive layer 46 to again cover portions of that adhesive layer, and to space portions of that adhesive layer and flexible material strip 18 at intermediate segment 24 apart from the skin of bridge 58 of user's nose 12. Such polyethylene disks may, for instance, be 0.015 inches in diameter and 0.005 inches thick. These polyethylene disks can be provided on adhesive layer 46′ through the use of a transfer drum, and again the size of the disks and their separation control the amount of adhesion reduction obtained.

Alternatively, as illustrated in FIG. 10, an adhesive material 46 at intermediate segment 24 can be covered by a perforated layer having the perforations controlled in size and in separation from one another to control the reduction of adhesion resulting from the use thereof on adhesive layer 46. One possibility is the use of a perforated polyethylene sheet, 48″, of 0.0005 to 0.0015 inches thickness to be positioned an adhesive layer 46 at intermediate segment 24 as shown in the fragmentary view of FIG. 10 of that segment illustrating such an alternative. Another possibility would be to apply a biocompatible varnish with suitable spaced openings therein over adhesive material layer 46.

Finally, the fragmentary view in FIG. 11 of intermediate segment 24 shows a further alternative in which a fine polyethylene powder, 45′, is used to cover more or less randomly positioned portions of adhesive layer 46. Here, the fineness of the polyethylene powder grains and the density by which they are applied to provide the desired average area of exposed adhesive 46 controls the resulting adhesion reduction obtained.

All of the above referenced patents; patent applications and publications are hereby incorporated by reference. Many variations of the present invention will suggest themselves to those of ordinary skill in the art in light of the above detailed description. All such obvious modifications are within the full-intended spirit and scope of the claims of the present application.

What is claimed is:

1. A nasal therapeutic system capable of introducing separating stresses in outer wall tissues of a section of human anatomy, comprising:
a truss of a single body having a pair of spaced-apart end surfaces which, if forced toward one another from initial positions to substantially reduce direct spacing therebetween by a spacing reduction force external to said truss, results in restoring forces in said truss tending to restore said direct spacing between said end surfaces;
ends engagement means adhered to said end surfaces and capable of engaging exposed surfaces of such outer wall tissues sufficiently to remain so engaged against said restoring forces;
an intermediate engagement means adhered to said truss at a surface thereof located between said end surfaces and capable of engaging exposed surfaces of human anatomy between the exposed surfaces of the outer wall tissues engaged by said ends engagement means but with less adhesion than that exhibited by said ends engagement means in engaging the outer wall tissues; and
an aromatherapy means.

2. The nasal therapeutic system of claim 1, wherein said intermediate engagement means is formed of an adhesive adhered to said truss and covered by a layer of material having openings extending therethrough.

3. The nasal therapeutic system of claim 2, wherein said layer is a polymer sheet.

4. The nasal therapeutic system of claim 2, wherein said layer is a polymer netting.

5. The nasal therapeutic system of claim 1, wherein said intermediate engagement means is formed of an adhesive adhered to said truss and covered with a film having openings extending therethrough.

6. The nasal therapeutic system of claim 5, wherein said film is a residue of a material deposition including a liquid.

7. The nasal therapeutic system of claim 1, wherein said intermediate engagement means is formed of an adhesive adhered to said truss and covered with a plurality of separated regions each containing a non-adhesive material.

8. The nasal therapeutic system of claim 7, wherein said non-adhesive material is a polymer material.

9. The nasal therapeutic system of claim 1, wherein said intermediate engagement means is formed of a plurality of separated regions each containing an adhesive adhered to said truss.

10. A nasal therapeutic system for preventing outer wall tissue of nasal passages of a nose from drawing in during breathing, comprising:
a unitary truss member having a normally, substantially planar state, the unitary truss member including:
a first end region adapted to engage the outer wall tissue of a first nasal passage;
a second end region adapted to engage the outer wall tissue of a second nasal passage;
an intermediate segment configured to traverse a portion of a nose located between the first and second nasal passages, the inherent tendency of the truss member to return to its normally planar state when flexed acting to stabilize the outer wall tissue and thereby prevent the outer wall tissue of the first and second nasal passages from drawing in during breathing;
a resilient member secured to at least a portion of the first and second end regions and the intermediate segment, the resilient member being capable, at least in part, of deformation that allows the truss member to conform to the outer wall tissue of the nasal passages of a nose; and
an aromatherapy means.

11. The nasal therapeutic system of claim 10 wherein the truss member includes:
a flexible strip of material defining the first and second end regions and the intermediate segment; and
said resilient member secured to a first side of the flexible strip of material by way of a deformable means, the resilient member defining the normally planar state of the truss member.

12. The nasal therapeutic system of claim 11, and further including:
an adhesive substance located on a second side of the flexible strip of material at the first and second end regions thereof for releasably securing the truss member to the outer wall tissue of the first and second nasal passages.

13. The nasal therapeutic system of claim 12, and further including:
first and second release liners covering the adhesive substance on the first and second end regions, respectively, of the flexible strip of material, the first and second release liners being readily removable from the flexible strip of material to expose the adhesive substance and permit the truss member to be secured to the outer wall tissue of the first and second nasal passages.

14. The nasal therapeutic system of claim 11 wherein the flexible strip of material is formed of an interwoven piece of fabric.

15. The nasal therapeutic system of claim 11, wherein the resilient member includes:
at least one resilient band oriented substantially parallel to a longitudinal extent of the flexible strip of material, the resiliency of the at least one resilient band acting to prevent the outer wall tissue of the first and second nasal passages from drawing in during breathing.

16. The nasal therapeutic system of claim 10, wherein the intermediate segment of the truss member includes an adhesive void, and wherein the truss member is configured to extend about a nose such that the intermediate segment traverses an exterior region of a bridge of a nose with the adhesive void located between the truss member and the bridge, the first end region engaging an exterior surface of the outer wall tissue of the first nasal passage and the second end region engaging an exterior surface of the outer wall tissue of the second nasal passage.

17. The nasal therapeutic system of claim 10, wherein the resilient member stabilizes the outer wall tissue by dilating the first and second nasal passages to thereby prevent the outer wall tissue of the first and second nasal passages from drawing in during breathing.

18. The nasal therapeutic system of claim 1 wherein the truss member is formed of a material that allows the skin of a nose to breathe to maximize comfort and minimize irritation.

19. A nasal therapeutic system capable of introducing separating stresses in nasal outer wall tissues, comprising:
a truss of a single body with a resilient member secured therein having a pair of spaced apart end surfaces which, if forced toward one another from initial positions to substantially reduce direct spacing therebetween by a spacing reduction force external to said truss, results in restoring forces in said truss tending to restore said direct spacing between said end surfaces;
engagement means adhered to said end surfaces and capable of engaging exposed surfaces of nasal outer wall tissues sufficiently to remain so engaged against said restoring forces; and
an aromatherapy means.

20. The nasal therapeutic system of claim 19, wherein said nasal therapeutic system is configured to restrain nasal outer wall tissues adjacent nasal passages therein from being drawn in during breathing, said truss having sufficient restoring forces to substantially maintain during inhalation that spacing occurring between said end surfaces prior to inhalation.

21. The nasal therapeutic system of claim 20, wherein said end surfaces are limited in separation therebetween so that, when said end surfaces are engaging nasal outer wall tissues adjacent nasal passages therein, a surface of said truss can be in contact with said nose for substantially all of that extent thereof between said end surfaces.

22. The nasal therapeutic system of claim 19, wherein said truss includes deformable material between exposed surfaces of any nasal outer wall tissues engaged by said engagement means and said resilient member.

23. The nasal therapeutic system of claim 22, wherein said single body of said truss further includes a flexible strip of said deformable material defining said pair of spaced apart end surfaces with said resilient member being secured to a first side of said flexible strip of deformable material.

24. The nasal therapeutic system of claim 19, wherein said truss and said engagement means are capable of being manually released from exposed surfaces of any nasal outer wall tissues engaged by said engagement means.

25. The nasal therapeutic system of claim 23, wherein said engagement means is an adhesive substance located on each of said pair of spaced apart end surfaces and capable of adhering to exposed surfaces of nasal outer wall tissues while adhering to said truss and yet permitting said truss and said engagement means to be manually released from exposed surfaces of any nasal outer wall tissues adhered to by said engagement means.

26. The nasal therapeutic system of claim 25, wherein said adhesive substance located on each of said pair of spaced apart surfaces is capable of adhering to a covering means while adhering to said truss prior to any engaging of exposed surfaces of nasal wall tissues yet permitting said covering means to be manually removed therefrom.

27. The nasal therapeutic system of claim 19, wherein said truss and said engagement means together are formed as a strip having a length substantially greater than either of its width and thickness, and a width substantially greater than its thickness everywhere along said length.

28. The nasal therapeutic system of claim 27, wherein said strip is of substantially constant thickness.

29. The nasal therapeutic system of claim 27, wherein said strip is substantially planar absent external forces applied thereto.

30. The nasal therapeutic system of claim 19, wherein said restoring forces in said truss arising if said end surfaces are forced adjacent to one another by said spacing reduction forces are sufficient, upon removal of said spacing reduction forces, to restore most of said direct spacing present between said end surfaces before application of such spacing reduction forces.

31. The nasal therapeutic system of claim 30, wherein said single body of said truss is of plastic construction.

32. The nasal therapeutic system of claim 19, wherein said spaced-apart end surfaces of said truss are terminated by end edges at opposite ends of said truss, and wherein said resilient member provides said restoring forces in said truss tending to restore said spacing between end surfaces thereof, said resilient member having opposite ends thereof each ending short of said end edges.

33. The nasal therapeutic system of claim 32, wherein said single body of said truss further includes a flexible strip of material defining said pair of spaced-apart end surfaces with said resilient member being deformably secured to a first side of said flexible strip of material.
